(19) United States
(12) Patent Application Publication
(10) Pub. No.: US 2003/0105443 A1
(43) Pub. Date: Jun. 5, 2003

(54) Absorbent article comprising mircroporous film with registration mark

(75) Inventors: Kazuyuki Ohnishi, Takaishi-City (JP); Hiroyuki Ogata, Nada-ku (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/338,394

(22) Filed: Jan. 8, 2003

Related U.S. Application Data
(63) Continuation of application No. PCT/US00/18725, filed on Jul. 10, 2000.

(51) Int. Cl. A61F 13/15

(52) U.S. Cl. 604/370; 604/372

(57) ABSTRACT

An absorbent article is disclosed. The absorbent article comprises a topsheet, a backsheet, and an absorbent core therebetween. The backsheet comprises a microporous film. The microporous film is provided with a mark for registration for processing registered graphics. The microporous film has a web modulus of not less than 160 gf/mm and a bending force of not more than 3.3 mgf·cm²/cm. The microporous film also comprises a material having a material modulus of not less than 310 gf/mm² at 3% strain.

### CROSS REFERENCE

This is a continuation of International Application PCT/US00/18725 filed on Jul. 10, 2000.

### TECHNICAL FIELD

This application relates to absorbent articles comprising a microporous film with a mark for registration. More specifically, the present application relates to absorbent articles comprising a microporous film with a mark for registration used for controlling and correcting the phase and position of simultaneously advancing continuous microporous webs.

### BACKGROUND

For disposable absorbent articles, e.g., disposable diapers, it is often desired to provide graphic designs registered (registered graphics) on the predetermined position of the absorbent articles to enhance their aesthetic appearance and their consumer acceptance. The positioning of a web pre-printed with registered graphics such that the graphics are properly placed in relation to the rest of the absorbent articles is desirable, e.g., in order to provide a large-sized graphics without cutting it at an incorrect location. Therefore, the web pre-printed with registered graphics is also provided a mark for registration used for controlling and correcting the phase and position of simultaneously advancing pre-printed continuous webs.

Such graphic designs and marks for registration are typically pre-printed on a part of layers that are positioned away from the wearer's body during use (often called “backsheet”) such that the wearer or the care taker can see graphic designs when in use of the absorbent article. Breathable polymer films that are particularly useful as backsheet materials for disposable absorbent articles typically have food surface characteristics that make them suitable for the application of multi-colored, high resolution graphics, which are consumer-preferred. Such an absorbent article comprising a microporous film with registered graphics is disclosed in, e.g., PCT publication WO 99/32164.

The backsheet provides a liquid impervious barrier so that exudates absorbed and contained in the absorbent core of the article are prevented from leaking, and particularly so that urine stains outside the diaper are prevented. The backsheet comprising microporous films also provides moisture permeability through the backsheet. The backsheet used for absorbent articles is also preferably soft and/or flexible. It has been known that softness and/or flexibility of the backsheet is obtainable by, e.g., decreasing the caliper and/or the basis weight of the film used for a backsheet. However, decreasing the caliper and/or the basis weight of the film is problematic because it also lowers a “web” modulus of the film. Because the film is typically tensioned for easiness of printing graphic designs and marks for registration on the film and/or joining to other members of the absorbent articles to assemble the absorbent articles, the low web modulus of the film contributes to creating significant variations of the strain, caused by the tensioning force during tensioning the film, on the film between each mark for registration. Such significant variations of the distance between each mark for registration could disable or at least deteriorate controlling and correcting the phase and position of advancing pre-printed continuous webs. Therefore, the film preferably has at least a level of web modulus not to cause significant variations of the strain on the film for the stable operation of a registered phasing system. However, it was practically difficult to maintain the level of the web modulus of the film while enhancing softness and/or flexibility of the film.

Based on the foregoing, there is a need for an absorbent article comprising a microporous film with a mark for registration which is soft and/or flexible while providing a web modulus sufficient for the stable operation of a registered phasing system. None of the existing absorbent articles provides all of the advantages and benefits of the present invention.

### SUMMARY

The present invention is directed to an absorbent article comprising a topsheet, a backsheet, and an absorbent core therebetween. The backsheet comprises a microporous film. The microporous film is provided with a mark for registration for processing registered graphics. The microporous film has a web modulus of not less than 160 gf/mm and a bending force of not more than 3.3 mgf·cm²/cm. The microporous film also comprises a material having a material modulus of not less than 310 gf/mm² at 3% strain.

These and other features, aspects, and advantages of the invention will become evident to those skilled in the art from a reading of the present disclosure.

### BRIEF DESCRIPTION OF THE DRAWINGS

While the specification concludes with claims particularly pointing out and distinctly claiming the invention, it is believed that the present invention will be better understood from the following description of preferred embodiments taken in conjunction with the accompanying drawings in which:

FIG. 1 is a back view of one embodiment of a disposable pull-on diaper with exemplary graphics and registration mark;

FIG. 2 is a simplified plan view of the pull-on diaper of FIG. 1 in its flat, uncontracted state prior to formation;

FIG. 3 is a schematic diagram showing simplified representations of a portion of a continuous diaper backsheet web having consecutively spaced graphics and registration mark printed thereon;

FIG. 4 is a schematic diagram of alternative embodiment of a continuous diaper backsheet web having consecutively spaced graphics and registration mark;

FIG. 5 is a schematic diagram of the first state for tensile stress measurement;

FIG. 6 is a schematic diagram of the second state for tensile stress measurement;

FIG. 7 is a cross sectional view taken along the line VII-VII shown in FIG. 6;

FIG. 8 is a schematic diagram of the third state for tensile stress measurement;

FIG. 9 is a cross sectional view taken along the line IX-IX shown in FIG. 8;

FIG. 10 is a schematic diagram of the forth state for tensile stress measurement;

FIGS. 11 and 12 are schematic diagrams of the bending property measurement; and

FIG. 13 is a graph showing the bending hysteresis curve.

### DETAILED DESCRIPTION

All references cited herein are incorporated herein by reference in their entireties. Citation of any reference is not an admission regarding any determination as to its availability as prior art to the claimed invention.

All percentages herein are by weight of compositions unless specifically stated otherwise. All ratios are weight ratios unless specifically stated otherwise. As used herein, the term “comprising” means that other steps and other ingredients which do not affect the end result can be added. This term encompasses the terms “consisting of” and “consisting essentially of.”

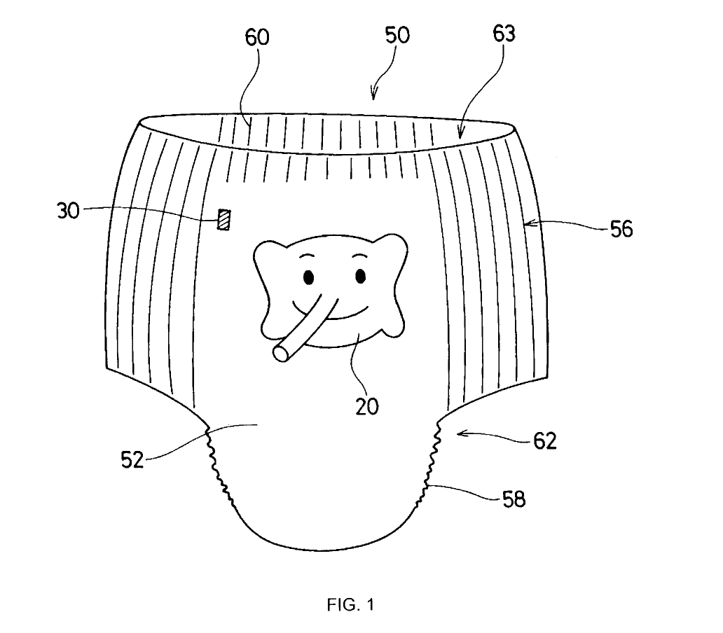

As used herein, the term “absorbent article” refers to devices which absorb and contain body exudates, and, more specifically, refers to devices which are placed against or in proximity to the body of the wearer to absorb and contain the various exudates discharged from the body. The term “disposable” is used herein to describe absorbent articles which are not intended to be laundered or otherwise restored or reused as an absorbent article (i.e., they are intended to be discarded after a single use and, preferably, to be recycled, composted or otherwise disposed of in an environmentally compatible manner). A preferred embodiment of an absorbent article of the present invention is the disposable absorbent article, pull-on diaper 50, shown in FIG. 1. The term “diaper” refers to an absorbent article generally worn by infants and incontinent persons that is worn about the lower torso of the wearer. The term “pull-on diaper” herein refers to pull-on garments worn by small children and other incontinent individuals to absorb and contain body exudates. It should be understood, however, that the present invention is also applicable to other absorbent articles such as incontinence briefs, incontinence undergarments, diaper holders and liners, feminine hygiene garments, training pants, and the like. It should further be understood that tape-type diapers are included herein.

The term “registered graphic” refers to single or multiple color graphic objects that are printed on a web close to a specified pitch length on a relaxed web basis. The term “mark for registration” or “registration mark” refers to a base mark to control and correct the phase and position of a web printed registered graphics.

The term “web” refers to a sheet-like material, such as film, woven, nonwoven, and/or combinations thereof.

Referring to FIG. 1, there is shown a preferred embodiment of a disposable pull-on diaper 50, which is generally pulled onto the body of the wearer by inserting the legs into the leg openings 62 and pulling the article up over the waist.

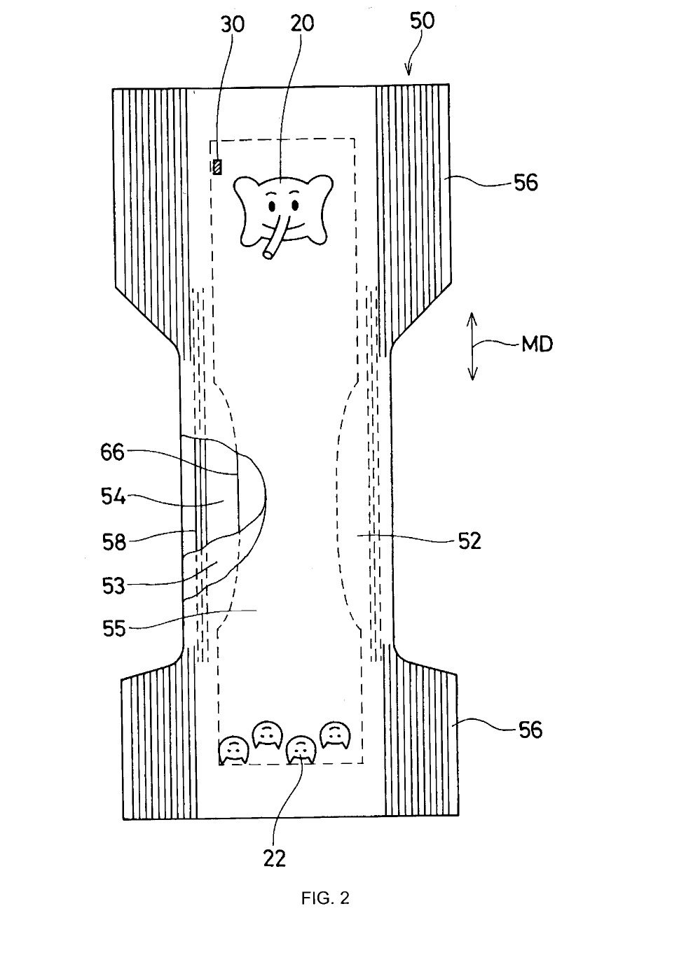

Referring to FIG. 2 as well, the diaper 50 generally comprises a backsheet 52, a topsheet 54 and an absorbent layer 66 located between the backsheet 52 and the topsheet 54. The topsheet 54 is located to be placed facing the body or nearest the body when the diaper is worn and is generally provided with a liquid permeable region so that body exudates can flow through the topsheet 54 to the absorbent layer 66. The backsheet 52, which is placed away from the body during wear, is typically liquid impermeable so that outer clothing or other articles are not wetted by the body exudates. Preferably, the backsheet 52 comprises a microporous polymer film 53 printed with registered graphics, as described herein. The backsheet 52 may further comprise a layer of nonwoven material 55 laminated to the microporous film 53, in which case there is provided a more cloth-like and garment-like feel than is typically obtained with a film backsheet only.

The diaper 50 has elastically extensible side panels 56 provided to ensure more comfortable and contouring fit by initially conformably fitting the pull-on diaper 50 to the wearer and sustaining this fit throughout the time of wear well past when it has been loaded with exudates. Leg elastics 58 and waist elastic region 60 are also provided to enhance the fit around the legs and waist, respectively. The side panels 56 are joined at seams to form a waist opening 63 and leg openings 62.

As will be understood by those of skill in the art, many other features for disposable absorbent articles are within the scope of the present invention. For example, barrier cuffs as described in Lawson and Dragoo U.S. Pat. Nos. 4,695,278 and 4,795,454 are a desirable feature for disposable absorbent articles. In addition, skin care-type topsheets that are provided with lotion thereon for the purpose of reducing skin irritation and chafing are a desirable feature herein.

FIG. 1 shows the back view of the diaper 50 with an exemplary registered graphic 20 and a registration mark 30 positioned in about the upper region of the backsheet, on the back side of the diaper 50. In FIG. 2, there is shown a simplified plan view of an embodiment of a disposable absorbent article in its flat, uncontracted state prior to formation. In this embodiment, the registered graphic 20 is shown in the back region of the diaper with registered graphics 22 additionally shown in the front region. The registered graphics 20 and 22 are positioned on the predetermined position of the diaper 50 such that the registered graphics 20 and 22 appear on the same position on each diaper without significant variation. Each diaper may be printed with the same pattern of the registered graphics. Alternatively, each diaper may be printed with two or more different patterns of the registered graphics.

The registered graphics 20, 22 and the registration mark 30 are printed on the microporous film 53 of the backsheet 52. The registered graphics 20, 22 may be printed on either side of the microporous film 53; i.e., the body facing side or the garment facing side as far as the registered graphics 20 and 22 are viewed when the diaper 50 is used. When the diaper 50 has the nonwoven material 55 laminated to the microporous film 53, the nonwoven material 55 preferably has transparency or at least translucency to the extent that the registered graphics 20 and 22 can be viewed through the nonwoven material 55.

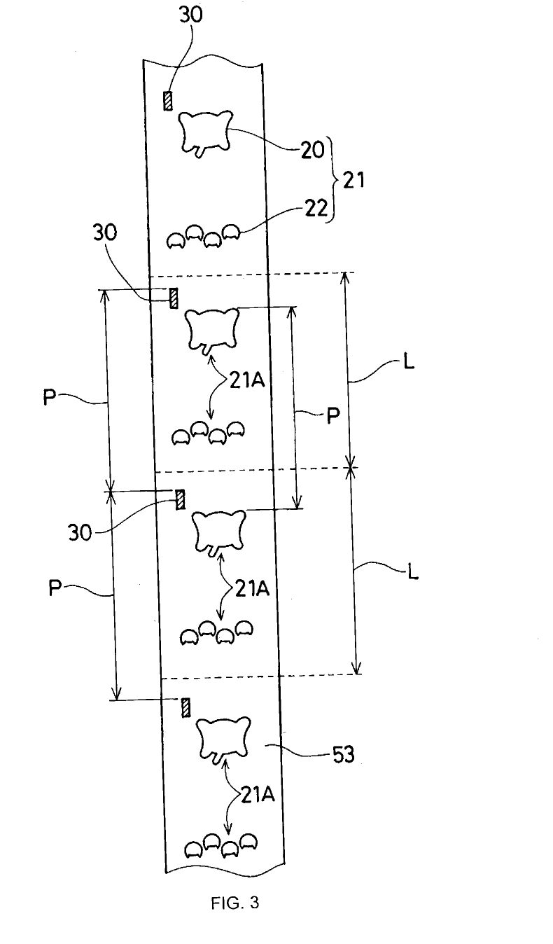

An exemplary portion of a microporous film web 53 printed with registered graphics 20 and 22 and registration marks 30 is shown in FIG. 3 (for the purpose of illustration, the registered graphics and registration mark are simplified and those sizes are modified). The microporous film web 53 in FIG. 3 is printed with four sets of graphics 21 comprising registered graphics 20 and 22 and a registration mark 30, each of which is separated by pitch length P. One set of graphics 21 comprising registered graphics 20 and 22 and registration mark 30 is printed to correspond to the full length L of one assembled diaper. In the embodiment shown in FIG. 3, the pitch length P is the same as the full length L of the diaper product. When the diaper is manufactured, the microporous film 53 is advanced on the manufacturing line such that the registered graphics 21 are phased with other members of the diaper such as a topsheet and an absorbent core and such that the registered graphics 21 appear on the same place of the diaper without significant variations.

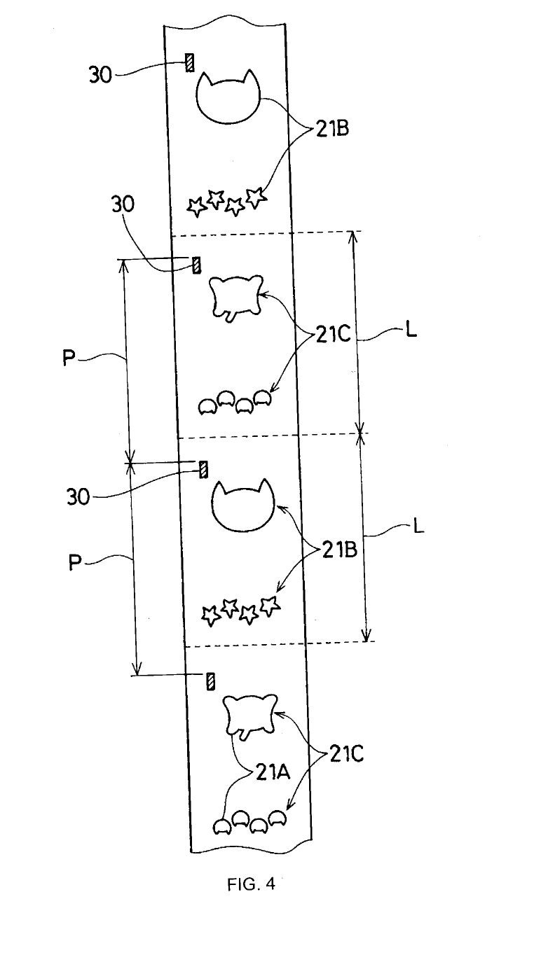

The graphics 21 may be of any shape, design, color or size, and that single or multiple designs may be used. The graphics 21 may be pre-printed on the microporous film 53. Alternatively pre-bonded, pre-applied, pre-cut, or pre-glued objects may be used. In the embodiment shown in FIG. 3, the graphics 21 comprises the registered graphics 20 and 22. Each pattern of the graphics 21 is the same throughout its entire length of the microporous film 53. Alternatively, the graphic 21 may comprise two or more of different graphic patterns such that each assembled diaper has different patterns of graphics 21. For example, the microporous film 53 of FIG. 4 has two different patterns of graphics 21B and 21C and each pattern of the graphic 21B and 21C is disposed alternately along the length of the microporous film 53. When each pattern of the graphics 21 is the same throughout its entire length as shown in FIG. 3, each graphic 21A is preferably separated by the pitch length P which is the same as the full length L of the diaper product. However, when the graphics 21 comprises two patterns of the graphics 21B and 21C as shown in FIG. 4, each graphic 21B and 21C is not necessarily separated by the same pitch length P (i.e., two consecutive pitch lengths between the graphics 21B and 21C and between the graphics 21C and 21B may be different) as far as each registration mark 30 maintains the same pitch length without significant variations.

Registration marks 30 are used so that the optical sensors of the combining and cutting mechanisms can detect the marks and thereby to properly align and to trim the film or film/nonwoven backsheet combination. Therefore, it is important to maintain a constant pitch length between each registration mark 30 and to minimize variations of the pitch length P for stably controlling and correcting the phase and position of advancing continuous webs printed with the registered graphics. The microporous film of the present invention accounts for such variations while providing softness and flexibility. The detail of the microporous film is described hereinbelow. While the registration mark 30 of the diaper shown in FIG. 1 is visible and discrete from the graphic 21, it is preferable that the registration mark 30 is a part of the graphic 21 such that the consumer does not recognize the presence of the registration mark while still being detectable by the machinery such as optical sensors. Alternatively, the registration mark 30 may be part of the finished product but invisible to the consumer. Alternatively, it may be sized and placed such that they are removed when the film is trimmed, so that they are not a part of the finished product and thus not visible to the consumer.

Referring to FIG. 2, the topsheet 54 and the backsheet 52 have length and width dimensions generally larger than those of the absorbent core 66. The topsheet 54 and the backsheet 52 extend beyond the edges of the absorbent core 66 to thereby form the periphery of the diaper 50. The topsheet 54, the backsheet 52, and the absorbent core 66 may be assembled in a variety of well known configurations.

The absorbent core 66 may be any absorbent member which is generally compressible, conformable, non-irritating to the wearer's skin, and capable of absorbing and retaining liquids such as urine and other certain body exudates. The absorbent core 66 may be manufactured in a wide variety of sizes and shapes (e.g., rectangular, hourglass, “T”-shaped, asymmetric, etc.) and from a wide variety of liquid-absorbent materials commonly used in disposable diapers and other absorbent articles such as comminuted wood pulp which is generally referred to as airfelt. Examples of other suitable absorbent materials include creped cellulose wadding; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials.

The configuration and construction of the absorbent core 66 may vary (e.g., the absorbent core may have varying caliper zones, a hydrophilic gradient, a superabsorbent gradient, or lower average density and lower average basis weight acquisition zones; or may comprise one or more layers or structures). Further, the size and absorbent capacity of the absorbent core 66 may also be varied to accommodate wearers ranging from infants through adults. However, the total absorbent capacity of the absorbent core 66 should be compatible with the design loading and the intended use of the diaper 50.

One embodiment of the diaper 50 has an asymmetric, modified T-shaped absorbent core 66 having ears in the front waist region but a generally rectangular shape in the rear waist region. Exemplary absorbent structures for use as the absorbent core 66 of the present invention that have achieved wide acceptance and commercial success are described in U.S. Pat. No. 4,610,678 entitled “High-Density Absorbent Structures” issued to Weisman et al. on Sep. 9, 1986; U.S. Pat. No. 4,673,402 entitled “Absorbent Articles With Dual-Layered Cores” issued to Weisman et al. on Jun. 16, 1987; U.S. Pat. No. 4,888,231 entitled “Absorbent Core Having A Dusting Layer” issued to Angstadt on Dec. 19, 1989; and U.S. Pat. No. 4,834,735, entitled “High Density Absorbent Members Having Lower Density and Lower Basis Weight Acquisition Zones”, issued to Alemany et al. on May 30, 1989. The absorbent core may further comprise the dual core system containing an acquisition/distribution core of chemically stiffened fibers positioned over an absorbent storage core as detailed in U.S. Pat. No. 5,234,423, entitled “Absorbent Article With Elastic Waist Feature and Enhanced Absorbency” issued to Alemany et al., on Aug. 10, 1993; and in U.S. Pat. No. 5,147,345, entitled “High Efficiency Absorbent Articles For Incontinence Management” issued to Young, LaVon and Taylor on Sep. 15, 1992. All of these patents are incorporated herein by reference.

The topsheet 54 is preferably positioned adjacent the inner surface of the absorbent core 28 and is preferably joined thereto and to the backsheet 52 by attachment means (not shown) such as those well known in the art. Suitable attachment means are described with respect to joining the backsheet 52 to the absorbent core 66. In a preferred embodiment of the present invention, the topsheet 54 and the backsheet 52 are joined directly to each other in the diaper periphery and are indirectly joined together by directly joining them to the absorbent core 66 by any suitable attachment means.

The topsheet 54 is preferably compliant, soft feeling, and non-irritating to the wearer's skin. Further, the topsheet 54 is preferably liquid pervious permitting liquids (e.g., urine) to readily penetrate through its thickness. A suitable topsheet 54 may be manufactured from a wide range of materials such as woven and nonwoven materials; polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be comprised of natural fibers (e.g., wood or cotton fibers), synthetic fibers (e.g., polymeric fibers such as polyester, polypropylene, or polyethylene fibers) or from a combination of natural and synthetic fibers. The topsheet 54 can be rendered hydrophilic by treating it with a hydrophilic finishing oil or a surfactant. Suitable methods for the treatment for the topsheet 54 include spraying the topsheet 54 material with surfactant and immersing the material into the surfactant. A more detailed discussion of such a treatment and hydrophilicity is contained in U.S. Pat. No. 4,988,344 entitled “Absorbent Articles with Multiple Layer Absorbent Layers” issued to Reising, et al. on Jan. 29, 1991 and U.S. Pat. No. 4,988,345 entitled “Absorbent Articles with Rapid Acquiring Absorbent Cores” issued to Reising on Jan. 29, 1991, each of which is incorporated by reference herein. Alternatively, the topsheet 24 may be a carded nonwoven material which is formed by fibers treated with hydrophilic finishing oil.

The backsheet 52 is that portion of the diaper 50 which is generally positioned away from the wearer's skin and which prevents the exudates absorbed and contained in the absorbent core 66 from wetting articles which contact the diaper 50 such as bedsheets and undergarments. Thus, the backsheet 52 is impervious to liquids (e.g., urine) and is preferably manufactured from a thin plastic film, although other soft, flexible liquid impervious materials may also be used. (As used herein, the term “flexible” refers to materials which are compliant and will readily conform to the general shape and contours of the human body.) While the backsheet 52 is impervious to liquids, the backsheet 52 permits moisture to escape from the diaper 50.

The moisture vapor transmission rate of the backsheet 52 is important in reducing the incidence of heat rash and other skin problems associated with high humidity conditions. In order to reduce humidity within the diaper, the backsheet 52 has a weighed average moisture vapor transmission rate of not less than about 40 g/m²/hr, preferably not less than about 80 g/m²/hr, more preferably not less than about 100 g/m²/hr. While the upper end of the moisture vapor transmission rate depends on a type of a material, and is selected in relation to the liquid impermeability/dampness of the backsheet, the moisture vapor transmission rate may be not more than about 1,000 g/m²/hr.

The moisture vapor transmission rate is measured by “Testing Methods of Water Vapour Permeability of Clothes—JIS L 1099” disclosed in Japanese Industrial Standard. While the method of JIS L 1099 specifies three separate independent test methods, Method A-1 (Calcium chloride method) can be specifically used.

The backsheet 52 is preferably positioned adjacent the outer surface of the absorbent core 66 and is preferably joined thereto by any suitable attachment means known in the art. For example, the backsheet 52 may be secured to the absorbent core 66 by a uniform continuous layer of adhesive, a patterned layer of adhesive, or an array of separate lines, spirals, or spots of adhesive. Adhesives which have been found to be satisfactory are manufactured by H. B. Fuller Company of St. Paul, Minn. and marketed as HL-1358J. An example of a suitable attachment means comprising an open pattern network of filaments of adhesive is disclosed in U.S. Pat. No. 4,573,986 entitled “Disposable Waste-Containment Garment”, which issued to Minetola et al. on Mar. 4, 1986. Another suitable attachment means comprising several lines of adhesive filaments swirled into a spiral pattern is illustrated by the apparatus and methods shown in U.S. Pat. No. 3,911,173 issued to Sprague, Jr. on Oct. 7, 1975; U.S. Pat. No. 4,785,996 issued to Ziecker, et al. on Nov. 22, 1978; and U.S. Pat. No. 4,842,666 issued to Werenicz on Jun. 27, 1989. Each of these patents is incorporated herein by reference. Alternatively, the attachment means may comprise heat bonds, pressure bonds, ultrasonic bonds, dynamic mechanical bonds, or any other suitable attachment means or combinations of these attachment means as are known in the art. Embodiments of the present invention are also contemplated wherein the absorbent core is not joined to the backsheet 52, and/or the topsheet 54 in order to provide greater extensibility in the front waist region and the rear waist region.

In the embodiment shown in FIGS. 1 and 2, the backsheet 52 comprises a breathable microporous film 53 and an outer sheet 55 which may comprise a nonwoven. The microporous film 53 positions adjacent the absorbent core 66 such that the microporous film 53 faces the absorbent core 66. Alternatively, another layer of material may be inserted between the absorbent core 66 and the microporous film 53. The nonwoven sheet 55 positions outwardly of the diaper.

The nonwoven outer sheet 55 may be joined with at least a portion of the garment-facing surface of the microporous film 53. Alternatively, the backsheet 52 may include any materials joined to the microporous film 53 such as woven webs, foams, scrims, loose fibers, or any other material or combination of materials known in the art that will give the diaper a cloth-like look and/or feel and is at a minimum air permeable. The nonwoven sheet 55 may cover all or substantially all of the garment-facing surface of the microporous film 53, or may cover only discrete predetermined portions. In a preferred embodiment, the nonwoven web of the nonwoven sheet 55 covers all or substantially all of the microporous film 53 in order to provide the diaper with a cloth-like look and feel. Further, the nonwoven sheet 55 may provide the diaper with a low cost landing zone capable of engaging the hooks of a hook and loop type fastener. (Such a landing zone could be utilized as a portion of a primary fastening system or as a means for disposing of a soiled diaper.)

The nonwoven web comprised in the nonwoven sheet 55 may comprise natural fibers (e.g. cotton or wood fibers), or may comprise fibers of polyethylene, polypropylene, polyester, or any combination of such fibers. Further, the nonwoven may be carded, spunbond, meltblown or air-through bonded or have any other characteristic or be manufactured in any manner known in the art. Preferably, the nonwoven is comprised of sufficient thermoplastic material to allow for thermal bonding of the material to other components of the diaper. An especially preferred nonwoven is a carded nonwoven made of 100% polypropylene fibers such as Sawabond 4111 manufactured by Vliesstofwerk Christian Heinrich Sandier GmbH & Co. KG, Germany.

The microporous film 53 may comprise any known material being moisture pervious and liquid impervious. For example, the microporous film 53 may comprise a breathable microporous film composed of a thermoplastic resin and inorganic fillers dispersed in the thermoplastic resin. Suitable thermoplastic polymers include polyolefins such as polyethylenes, including liner low density polyethylene (LLDPE), low density polyethylene (LDPE), ultra low density polyethylene (ULDPE), high density polyethylene (HDPE), or polypropylene and blends thereof with the above and other materials. Examples of other suitable thermoplastic polymers which may also be used include, but are not limited to, polyester, polyurethanes, compostable or biodegradable polymers, thermoplastic elastomers, and metallocene catalyst-based polymers (e.g., INSITE® available from Dow Chemical Company and Exxact® available from Exxon). The inorganic material or filler is selected from the group consisting of calcium carbonate, clay and titanium dioxide, with the preferred inorganic filler being calcium carbonate. Preferably, the microporous film 53 may comprise polyethylene of from about 30 weight % to about 55 weight % and preferably from about 40 weight % to about 45 weight %; and CaCO₃ of from about 45 weight % to about 70 weight % and preferably from about 55 weight % to about 60 weight %.

The inorganic filler and the thermoplastic polymer are blended together to form a homogeneous mixture in a suitable mixing extruder, or in a separate preliminary compounding step. The mixture is then cast or blown into a film. The obtained film is stretched at least in one direction to impart breathability on the substantially entire area of the film. The step of stretching a film to impart breathability may be done at a different place prior to manufacturing process of absorbent articles. Alternatively, the step of stretching may be done at the same place, i.e., same manufacturing process, prior to assembling a breathable microporous film with other elements of absorbent articles. In any cases, the film is imparted breathability on the substantially entire area of the film before the resulting breathable microporous film is assembled with other elements of absorbent articles.

The microporous film of the present invention should have desirable properties that are beneficial in the processing of the film in connection with the manufacture of absorbent articles with registered graphics and in the use of absorbent articles. The desirable properties of the microporous film of the present invention are web modulus of the microporous film, bending force value of the microporous film, and material modulus of the microporous film. The microporous film may have other desirable physical properties as described below.

Web modulus of the microporous film is important to the stable operation of a registered graphics phasing system. As used herein, “web modulus” means the mechanical property defined as the slope of the straight line which is connected between 1% strain and 5% strain in a material's stress/strain curve. Web modulus can be measured by the method described herein below. The modulus determines the amount of tension required to force an adjustment in the phase position of the film during processing. Microporous films of the present invention have a web modulus of not less than about 160 gf/mm. While the upper limit of the web modulus of the microporous films may be determined arbitrarily by the skilled in the art, the web modulus of the microporous films may be not more than 510 gf/mm, preferably not more than 300 gf/mm. The microporous films having a web modulus above do not cause significant variations of strain in the film or at least reduce the variation of the strain while the film is tensioned for being printed with graphic designs. The distances between the registration mark on the film remains relatively constant or at least remains the distance with a variation which is controllable by the system of handling the film. Therefore, the microporous film of the present invention is desirable for a registered graphics phasing system which is controlled based on the signal from the registration mark on the film.

Bending force of the microporous film is important for providing consumers, i.e., caregiver and wearer, with softness and/or flexibility of the outer surface of the absorbent article. As used herein, “bending force” means the mechanical property defined as the slope of M-K curve shown in FIG. 13. M is bending momentum per unit width and K is curvature. Bending force can be measured by the method described herein below. The microporous film of the present invention may have an average bending force value of not more than about 3.3 mgf·cm²/cm, preferably not more than about 3.0 mgf·cm²/cm, and more preferably not more than about 2.7 mgf·cm²/cm. The lower limit of the bending force may be determined arbitrarily by the skilled in the art.

It has been known that it is possible to obtain relatively low bending force film by decreasing caliper of the film and/or basis weight of the film. However, decreasing caliper of the film and/or basis weight of the film results in lowering the web modulus of the film, thereby causing an unstable operation of a registered graphics phasing system. Therefore, it is important in the present invention that the material modulus of the microporous film is maintained in a desirable range. As used herein, “material modulus” means the mechanical property defined as the tensile stress at 3% strain per the unit cross sectional area of the film. Material modulus can be determined by the method described herein below. Material modulus of the microporous film of the present invention is not less than 310 gf/mm² at 3% strain, preferably not less than 330 gf/mm² at 3% strain, more preferably not less than 350 gf/mm² at 3% strain. Such material modulus of the microporous film allows the microporous film to maintain the range of the web modulus above while decreasing caliper or basis weight of the film, i.e., providing desirable bending force for softness and/or flexibility. The desirable range of material modulus can be achieved by, e.g., adjusting the molecular structure of the thermoplastic resin of the microporous film. For example, such an adjustment may be achieved by increasing the number and/or size of crystallization (e.g. lamella structure) in the polymer of the thermoplastic resin. Without being wished to be bound by the theory, it is believed that the more crystallization makes the molecular solidity of the resin to be increased, thereby increasing the material modulus of the film. More solidity of the non-crystallization area also may lead to larger material modulus of the film. The adjustment may be also achieved by arranging the orientation of the polymer. Aligning the orientation of the polymer leads to higher material modulus in the direction. More concretely, higher crystallization and more lamella structure tend to promote the polymer's orientation in the machine direction when the film is being stretched in the film making process. Usually, both the side chains of LLDPE (linear low density polyethylene) and modifier (normally, lower molecular weight polymer) in the resin are obstructers of the crystallization and lamella structure of the polymer. Therefore, it is possible to control such molecular structure of the resin by selecting the resin formula.

Basis weight refers to the weight of one square meter of planar web material. Generally, higher basis weight leads to larger web modulus which has a good effect on the process feasibility of the film, but it also leads to less softness and/or flexibility of the film. Exemplary basis weight herein is preferably not more than 35 grams per square meter (gsm). While the lower limit of the basis weight of the microporous films may be determined arbitrarily by the skilled in the art, the basis weight of the microporous films may be not less than 15 gsm.

Film caliper refers to the thickness of the film. Exemplary film caliper is preferably 45 μm. While the lower limit of the film caliper of the microporous films may be determined arbitrarily by the skilled in the art, the film caliper of the microporous films may be not less than 15 μm.

The films of the present invention also preferably have other desirable properties that are beneficial in the processing of the film in connection with the manufacture of absorbent articles. Although it will be understood by those of skill in the art that the films of the present invention have many uses, for purposes of illustration the following description will focus upon use of the film as a backsheet for a disposable diaper.

Suitable microporous film is supplied from Mitsui Chemical, Japan under the name of Espoir PG-P2 25 gsm. This film has web modulus of 233 gf/mm, material modulus of 390 gf/mm² at 3% strain, and bending force of 2.5 mgf·cm²/cm. The film also has a basis weight of 25 gsm, and a caliper of 23 mm. Such a film is preferable as a microporous film with a registration mark for a registered graphics phasing system.

### METHOD

Method to Measure Web Modulus and Material Modulus

Web modulus and material modulus of a test web are measured as follows.

1. Measurement of Tensile Stress of Web

Sample Preparation

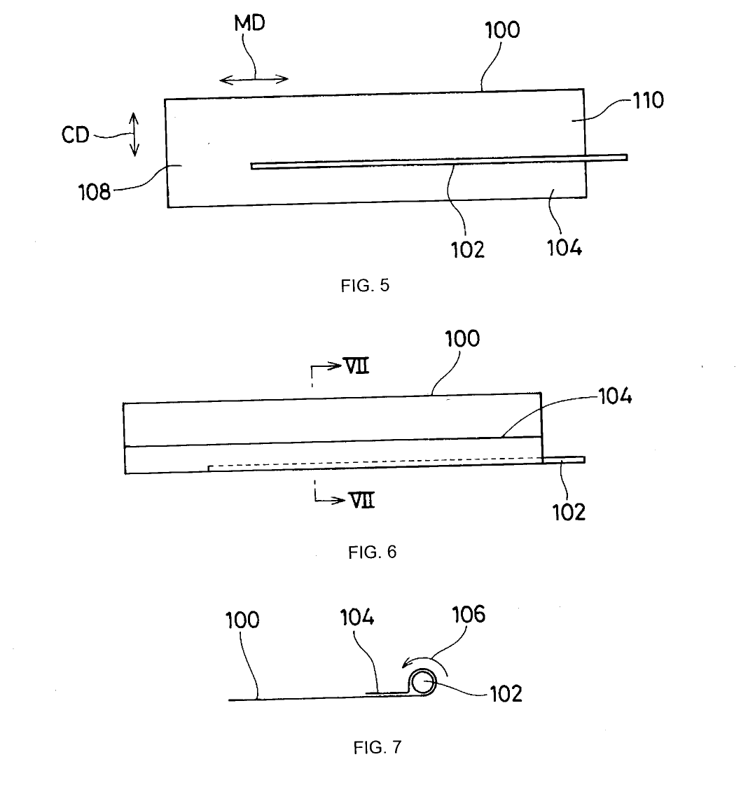

1. Cut a test web into a test piece 100 of 610 mm length in the machine direction (MD) and 150 mm width, place the test piece on a flat table and smooth out flat. (FIG. 5)

2. Place the 9.5 mm diameter steel rod 102 on the test piece 100 in the MD such that its location in the cross direction (CD) should be in about ⅓ the way (about 50 mm) from one longitudinal side 104 of the test piece 100. The rod 102 should stick out of the test piece with about 2.5 cm so that it would be easier to remove it later. (FIG. 5)

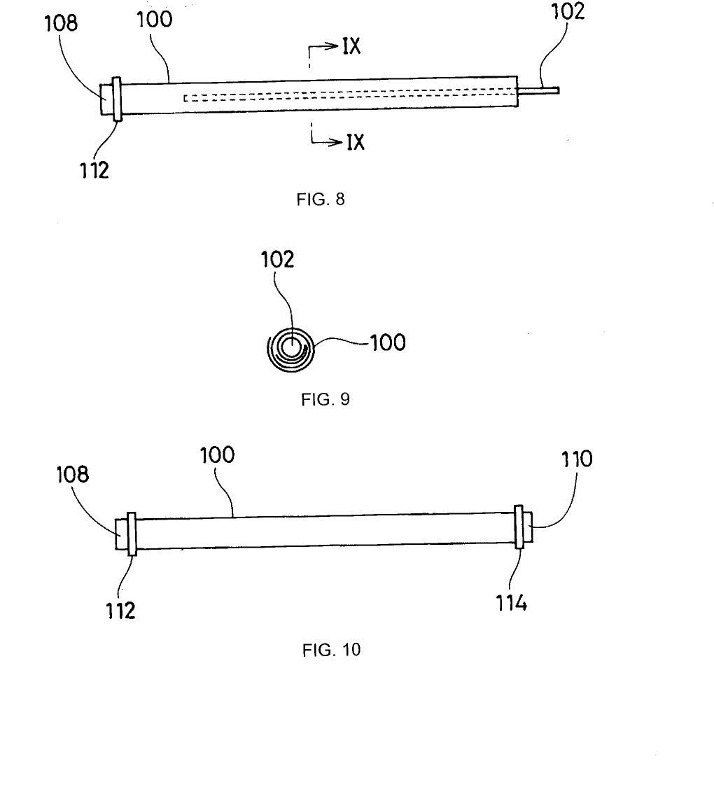

3. Fold the side of one longitudinal side 104 of the test piece 100 over the rod 102 along the rod 102 and lay flat (FIG. 6). And then, tuck the test piece 100 around the rod 102 in the CD as shown by the arrow 106 (FIG. 7) and roll up the test piece 100. (FIGS. 8 and 9)

4. Be as careful as possible to avoid wrinkles in the test piece 100 and keep the rod parallel to the longitudinal side of the test piece 100. Flatten the first end edge 108 of the rolled test piece 100 in which the rod 102 is not present. Staple the flattened first end edge 108 several times (staple at the first end edge 108 is designated by the reference number of 112 in FIG. 8) through the multiple layers of the test piece 100 to join these layers so that they will not slip in during the test.

5. Pull the rod out of the second end edge 110 carefully not to let the test piece unwind. Flatten the second end edge 110 such that it is flat in the same plane as the first end edge 108, and staple it several times (staple at the second end edge 110 is designated by the reference number of 114 in FIG. 10) such that the distance between the staples at the first end edge 108 and the second end edge 110 is about 560 mm which is sufficient for the gage length (508 mm) of the tensile tester below. Thus, a test sample is prepared.

Instrument Set Up

The instrument (Tensile tester:Instron 5564/MTS, Testworks ver.3.06) is set up to pull the test samples under the following condition.

| | |
|---|---|
| Master method | Tensile |
| Load cell | 100 N |
| Gauge length | 508 mm |
| Cross head speed | 254 mm/minute |
| Points for reading stress | at 1%, 3%, 5% strain |

Measurement

The measurement is made according to the following procedure.

1. Insert one end edge of a test sample into the upper jaw of tensile tester and close it.

2. Align the strip between the upper and lower jaws.

3. Place the other end edge of the test sample into the lower jaw with enough tension to eliminate any slacks.

4. Reset the tension of the cross head (Load meter) of the tensile tester.

5. Close the lower jaw with confirming that the load meter does not indicate more than 5.0 g.

6. Start particular tester.

From the measurement above, tensile stresses of the test sample at 1%, 3% and 5% strain are measured.

2. Measurement of Caliper of Web

The measurement of caliper of a test web is made by using the caliper gauge ‘EG-225’, made by Ono Sokki, Japan. The measurement is made under the conditions of 7.0 gf/cm² of pressure with the round contact plate which area is 400 πmm², according to the following procedure.

1. Make sure the contact plate fixed and turn the power switch on.

2. Select unit for mm.

3. Make sure no dust or other objects on the stand and under the plate, then push the reset button.

4. Put a test web between the plate and the stand slowly.

5. After several seconds, when the number in the meter is stabilized, record the value.

3. Calculation of Web Modulus

Tensile stresses at 1% and 5% strain are measured with the test method above. The differential of these tensile stresses per unit width is calculated, and then, the differential is divided by 0.04 which means the differential of 5% and 1%. This value is reported as web modulus for each specimen.

Web modulus(gf/mm)=(Tensile stress(gf)at 5% strain−Tensile stress(gf)at 1% strain)/((0.05−0.01)·150(mm))

wherein 150 (mm) is the width of the test piece.

4. Calculation of Material Modulus

Tensile stress at 3% strain is measured with the test method above. This value divided by cross section area is calculated and reported as material modulus for each specimen.

Material modulus(gf/mm²)=Tensile stress(gf)at 3% strain/(150 mm·Thickness(mm))

wherein 150 (mm) is the width of the test piece.

Method to Measure Bending Force

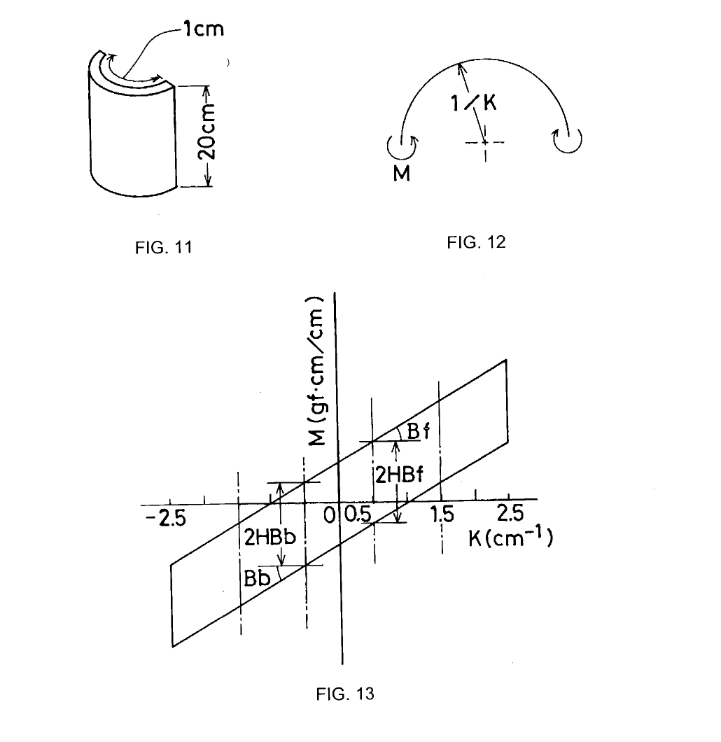

A bending tester, KES-FB2, Kato Tech. Co Ltd., is used to measure bending force. The deformation mode is a pure bending between the curvature K=−2.5 cm⁻¹ and 2.5 cm⁻¹. The effective dimension for the measurement is 20 cm in length and 1.0 cm in width (rectangular). The specimen is bent as shown in FIGS. 11 and 12. The bending rate is 0.5 cm⁻¹/sec. As a result, the bending hysteresis curve as shown in FIG. 13 is obtained by the measurement. The horizontal axis shows the curvatures K cm⁻¹ and the vertical axis shows the bending moment per unit width M (gf·cm/cm). The bending force is calculated as follows:

Bending Force=(Bf+Bb)/2

where Bf and Bb are the slopes of the hysteresis curves between K=0.5 cm⁻¹ and 1.5 cm⁻¹ and K=−0.5 cm⁻¹ and −1.5 cm⁻¹ respectively.

Measurements are carried out in the MD and CD directions of the same web specimen. The average bending force is the mean value of the above bending force obtained from the measurements about the MD and CD directions of the specimen.

It is understood that the examples and embodiments described herein are for illustrative purposes only and that various modifications or changes in light thereof will be suggested to one skilled in the art without departing from the scope of the present invention.

What is claimed is:

1. An absorbent article comprising a topsheet, a backsheet, and an absorbent core therebetween, the backsheet comprising a microporous film, the microporous film provided with a mark for registration, wherein
the microporous film has a web modulus of not less than 160 gf/mm and a bending force of not more than 3.3 mgf·cm²/cm, and the microporous film comprises a material having a material modulus of not less than 310 gf/mm² at 3% strain.

2. The absorbent article of claim 1 wherein the material of the microporous film comprises a thermoplastic resin and inorganic fillers.

3. The absorbent article of claim 1 wherein the microporous film has a moisture vapor transmission rate of not less than 40 g/m²/hr.

4. The absorbent article of claim 3 wherein the microporous film has a material modulus of not less than 330 gf/mm² at 3% strain.

5. The absorbent article of claim 4 wherein the microporous film has a material modulus of not less than 350 gf/mm² at 3% strain.

6. The absorbent article of claim 5 wherein the microporous film has a bending force of not more than 3.0 mgf·cm²/cm.

7. The absorbent article of claim 6 wherein the microporous film has a basis weight in the range of 15 g/m² and 35 g/m².

8. The absorbent article of claim 1 wherein the microporous film is printed with graphics disposed associated with the mark for registration.

9. The absorbent article of claim 8 wherein the graphics have two or more of different graphics patterns, each of the graphics pattern is disposed associated with each mark for registration.
